(19) United States
(12) Patent Application Publication
(10) Pub. No.: US 2023/0407254 A1
(43) Pub. Date: Dec. 21, 2023

(54) INDUCIBLE PLURIPOTENT STEM CELL DERIVED REGENERATIVE T CELLS

(71) Applicant: CREATIVE MEDICAL TECHNOLOGIES, INC., Phoenix, AZ (US)

(72) Inventors: Thomas ICHIM, San Diego, CA (US); Amit PATEL, Salt Lake City, UT (US); Courtney BARTLETT, Niceville, FL (US)

(73) Assignee: CREATIVE MEDICAL TECHNOLOGIES, INC., Phoenix, AZ (US)

(21) Appl. No.: 18/331,098

(22) Filed: Jun. 7, 2023

Related U.S. Application Data
(60) Provisional application No. 63/353,011, filed on Jun. 16, 2022.

(51) Int. Cl. C12N 5/0783

(52) U.S. Cl. CPC C12N 5/0636; C12N 2506/45; C12N 2501/2307; C12N 2501/2302; C12N 2501/999; C12N 2500/98

(57) ABSTRACT

Disclosed are novel cellular compositions of matter and treatment means for generation of universal donor regenerative T cells by exposure to mesenchymal stem cells or supernatant derived thereof. In one embodiment, regenerative T cells are created by differentiation of pluripotent stem cells in the presence of supernatant generated from activated mesenchymal stem cell population. The invention provides for creation of T cells which are capable of endowing regenerative activity, and/or anti-inflammatory, and/or angiogenic activity.

### CROSS-REFERENCE TO RELATED APPLICATIONS

This application claims priority to U.S. Provisional Application No. 63/353,011 titled “Inducible Pluripotent Stem Cell Derived Regenerative T Cells” filed Jun. 16, 2022, which is hereby incorporated by reference herein in its entirety.

### FIELD OF THE INVENTION

The invention relates to methods of producing enhanced regenerative T cells.

### BACKGROUND OF THE INVENTION

Stem cell therapy is currently limited by pulmonary blockade of intravenously injected mesenchymal cells, in part due to the large size of the administered cells. Additionally, donor to donor variability has historically caused inability to reproduce clinical results despite cells appearing to have identical visional and phenotypic characteristics. The current invention seeks to overcome existing limitations by creation of “universal donor” regenerative T cells from standardized but activated pluripotent stem cells.

### SUMMARY

Preferred embodiments include methods for production of T cells possessing ability to regenerate injured tissue comprising the steps of: a) obtaining a pluripotent stem cell; b) differentiating said pluripotent stem cell into a T cell; c) contacting said pluripotent stem cell at one or more time points during the differentiation process with mesenchymal stem cells or products derived thereof; and d) isolating T cells possessing regenerative activity.

Preferred methods include embodiments wherein said pluripotent stem cells are selected from a group of cells comprising of: a) inducible pluripotent stem cells; b) somatic cell nuclear transfer derived stem cells; c) embryonic stem cells; and d) parthenogenic derived stem cells.

Preferred methods include embodiments wherein said pluripotent stem cells are differentiated into T cells by sequential culture in cytokines and conditions replicating thymopoiesis.

Preferred methods include embodiments wherein said culture conditions comprise of: a) de-aggregating pluripotent stem cells; b) treating said cells with interleukin-7 for 1-7 days at a concentration of 0.1-100 pg/ml; c) subsequently treating said cells with interleukin-2 and interleukin-7 for an additional 1-14 days; d) optionally contacting the cells in step “c” with agonistic antibody to CD3 at a sufficient concentration to induce phosphorylation of TCR zeta chain; and e) extracting non-adherent cells from the culture.

Preferred methods include embodiments wherein said embryonic stem cell population expresses genes selected from a group comprising of: stage-specific embryonic antigens (SSEA) 3, SSEA 4, Tra-1-60 and Tra-1-81, Oct-3/4, Cripto, gastrin-releasing peptide (GRP) receptor, podocalyxin-like protein (PODXL), Rex-1, GCTM-2, Nanog, and human telomerase reverse transcriptase (hTERT).

Preferred methods include embodiments wherein said inducible pluripotent stem cell possesses markers selected from a group comprising of: CD10, CD13, CD44, CD73, CD90, PDGFr-alpha, PD-L2, and HLA-A, -B, -C and possesses ability to undergo at least 40 doublings in culture, while maintaining a normal karyotype upon passaging up to 50 times.

Preferred methods include embodiments wherein said parthenogenic stem cells wherein said parthenogenically derived stem cells are generated by addition of a calcium flux inducing agent to activate an oocyte followed by enrichment of cells expressing markers selected from a group comprising of SSEA-4, TRA 1-60 and TRA 1-81.

Preferred methods include embodiments wherein said somatic cell nuclear transfer derived stem cells possess a phenotype negative for SSEA-1 and positive for SSEA-3, SSEA-4, TRA-1-60, TRA-1-81, and alkaline phosphatase.

Preferred methods include embodiments wherein said mesenchymal stem cell are derived from tissue comprising a group selected from: a) Wharton's Jelly/umbilical cord tissue; b) bone marrow; c) peripheral blood; d) mobilized peripheral blood; e) endometrium; f) hair follicle; g) deciduous tooth; h) testicle; i) adipose tissue; j) skin; k) amniotic fluid; l) cord blood; m) omentum; n) muscle; o) amniotic membrane; o) periventricular fluid; and p) placental tissue.

Preferred methods include embodiments wherein said mesenchymal stem cells express a marker or plurality of markers selected from a group comprising of: STRO-1, CD90, CD73, CD105, CD54, CD106, HLA-I markers, vimentin, ASMA, collagen-1, fibronectin, LFA-3, ICAM-1, PECAM-1, P-selectin, L-selectin, CD49b/CD29, CD49c/CD29, CD49d/CD29, CD61, CD18, CD29, thrombomodulin, telomerase, CD10, CD13, STRO-2, VCAM-1, CD146, and THY-1.

Preferred methods include embodiments wherein said mesenchymal stem cells do not express substantial levels of HLA-DR, CD117, and CD45.

Preferred methods include embodiments wherein said mesenchymal stem cells express CD56.

Preferred methods include embodiments wherein said mesenchymal stem cell are activated by exposure to a toll like receptor agonist

Preferred methods include embodiments wherein said toll like receptor is TLR-1.

Preferred methods include embodiments wherein said activator of TLR-1 is Pam3CSK4.

Preferred methods include embodiments wherein said toll like receptor is TLR-2.

Preferred methods include embodiments wherein said activator of TLR-2 is HKLM.

Preferred methods include embodiments wherein said toll like receptor is TLR-3.

Preferred methods include embodiments wherein said activator of TLR-3 is Poly:IC.

Preferred methods include embodiments wherein said toll like receptor is TLR-4.

Preferred methods include embodiments wherein said activator of TLR-4 is LPS.

Preferred methods include embodiments wherein said activator of TLR-4 is Buprenorphine.

Preferred methods include embodiments wherein said activator of TLR-4 is Carbamazepine.

Preferred methods include embodiments wherein said activator of TLR-4 is Fentanyl.

Preferred methods include embodiments wherein said activator of TLR-4 is Levorphanol.

Preferred methods include embodiments wherein said activator of TLR-4 is Methadone.

Preferred methods include embodiments wherein said activator of TLR-4 is Cocaine.

Preferred methods include embodiments wherein said activator of TLR-4 is Morphine.

Preferred methods include embodiments wherein said activator of TLR-4 is Oxcarbazepine.

Preferred methods include embodiments wherein said activator of TLR-4 is Oxycodone.

Preferred methods include embodiments wherein said activator of TLR-4 is Pethidine.

Preferred methods include embodiments wherein said activator of TLR-4 is Glucuronoxylomannan from Cryptococcus.

Preferred methods include embodiments wherein said activator of TLR-4 is Morphine-3-glucuronide.

Preferred methods include embodiments wherein said activator of TLR-4 is lipoteichoic acid.

Preferred methods include embodiments wherein said activator of TLR-4 is (3-defensin 2.

Preferred methods include embodiments wherein said activator of TLR-4 is small molecular weight hyaluronic acid.

Preferred methods include embodiments wherein said activator of TLR-4 is fibronectin EDA.

Preferred methods include embodiments wherein said activator of TLR-4 is snapin.

Preferred methods include embodiments wherein said activator of TLR-4 is tenascin C.

Preferred methods include embodiments wherein said toll like receptor is TLR-5.

Preferred methods include embodiments wherein said activator of TLR-5 is flagellin.

Preferred methods include embodiments wherein said toll like receptor is TLR-6.

Preferred methods include embodiments wherein said activator of TLR-6 is FSL-1.

Preferred methods include embodiments wherein said toll like receptor is TLR-7.

Preferred methods include embodiments wherein said activator of TLR-7 is imiquimod.

Preferred methods include embodiments wherein said toll like receptor of TLR-8.

Preferred methods include embodiments wherein said activator of TLR8 is ssRNA40/LyoVec.

Preferred methods include embodiments wherein said toll like receptor of TLR-9.

Preferred methods include embodiments wherein said activator of TLR-9 is a CpG oligonucleotide.

Preferred methods include embodiments wherein said activator of TLR-9 is ODN2006.

Preferred methods include embodiments wherein said activator of TLR-9 is Agatolimod.

Preferred methods include embodiments wherein the cells are derived in xenofree media.

Preferred methods include embodiments wherein the cells are delivered in platelet rich plasma/platelet lysate carrier solution.

Preferred methods include embodiments wherein the cells are functionally activated with a trivalent gene construct.

Preferred methods include embodiments wherein the cells are functionally activated with a polyvalent gene construct.

### DETAILED DESCRIPTION OF THE INVENTION

The invention provides utilization of pluripotent stem cells to generate T cells possessing regenerative activity through in vitro differentiation of T cells in the presence of activated mesenchymal stem cells and/or products derived thereof.

“Pluripotency” is defined as cells capable of differentiating into the lymphoid lineage, more specifically into the T cell lineage. Cell markers for pluripotent stem cells include but are not limited to: alkaline phosphatase, Oct-4, Nanog, Stage-specific embryonic antigen-3 (SSEA-3), Stage-specific embryonic antigen-4 (SSEA-4), TRA-1-60, TRA-1-81, TRA-2-4916E, Sox2, growth and differentiation factor 3 (GDF3), reduced expression 1 (REX1), fibroblast growth factor 4 (FGF4), embryonic cell-specific gene 1 (ESG1), developmental pluripotency-associated 2 (DPPA2), DPPA4, telomerase reverse transcriptase (hTERT), SALL4, E-CADHERIN, Cluster designation 30 (CD30), Cripto (TDGF-1), GCTM-2, Genesis, Germ cell nuclear factor, and Stem cell factor (SCF or c-Kit ligand).

“Potency”, as used herein, refers broadly to the concentration, e.g., molar, of a reagent (such as hemangioblast-derived MSCs) that produces a defined effect. Potency may be defined in terms of effective concentration (EC50), which does not involve measurements of maximal effect but, instead, the effect at various locations along the concentration axis of dose response curves. Potency may also be determined from either graded (EC50) or quantal dose-response curves (ED50, TD50 and LD50); however, potency is preferably measured by EC50. The term “EC50” refers to the concentration of a drug, antibody or toxicant which induces a response halfway between the baseline and maximum effect after some specified exposure time. The EC50 of a graded dose response curve therefore represents the concentration of a compound where 50% of its maximal effect is observed. The EC50 of a quantal dose response curve represents the concentration of a compound where 50% of the population exhibit a response, after a specified exposure duration. The EC50 may be determined using animal studies in which a defined animal model demonstrates a measurable, physiological change in response to application of the drug; cell-based assays that use a specified cell system, which on addition of the drug, demonstrate a measureable biological response; and/or enzymatic reactions where the biological activity of the drug can be measured by the accumulation of product following the chemical reaction facilitated by the drug. Preferably, an immune regulatory assay is used to determine EC50. Non-limiting examples of such immune regulatory assays include intracellular cytokine, cytotoxicity, regulatory capacity, cell signaling capacity, proliferative capacity, apoptotic evaluations, and other assays.

“Mesenchymal stem cells” (MSC) as used herein refers to multipotent stem cells with self-renewal capacity and the ability to differentiate into osteoblasts, chondrocytes, and adipocytes, among other mesenchymal cell lineages. In addition to these characteristics, MSCs may be identified by the expression of one or more markers as further described herein. Such cells may be used to treat a range of clinical conditions, including immunological disorders as well as degenerative diseases such as graft-versus-host disease (GVHD), myocardial infarction and inflammatory and autoimmune diseases and disorders, among others. Except where the context indicates otherwise, MSCs may include cells from adult sources and cord blood. MSCs (or a cell from which they are generated, such as a pluripotent cell) may be genetically modified or otherwise modified to increase longevity, potency, homing, or to deliver a desired factor in the MSCs or cells that are differentiated from such MSCs. As non-limiting examples thereof, the MSCs cells may be genetically modified to express Sirt1 (thereby increasing longevity), express one or more telomerase subunit genes optionally under the control of an inducible or repressible promoter, incorporate a fluorescent label, incorporate iron oxide particles or other such reagent (which could be used for cell tracking via in vivo imaging, MRI, etc., see Thu et al., Nat Med. 2012 Feb. 26; 18(3):463-7), express bFGF which may improve longevity (see Go et al., J. Biochem. 142, 741-748 (2007)), express CXCR4 for homing (see Shi et al., Haematologica. 2007 July; 92(7):897-904), express recombinant TRAIL to induce caspase-mediated apoptosis in cancer cells like Gliomas (see Sasportas et al., Proc Natl Acad Sci USA. 2009 Mar. 24; 106(12):4822-7), etc.

“Phenotype” designates the presence or absence of a particular marker, especially at the cell surface, or a set of cells within the population. For example, the “CD34+CD43+” phenotype designates a cell, or a set of cells in the population, which express CD34 and CD43. “CD43−” designates a cell, or a set of cells in the population, which does not express CD43. CD43, also known as Ly-48, leucosialin, sialophorin, leukocyte sialoglycoprotein, and W3/13, is a type I transmembrane glycoprotein comprising numerous O-glycosylation and sialylation sites. The phenotype can be identified by means known in the art. For example, an antibody specific for CD43 conjugated to allophycocyanin (APC) may be used to measure the expression of CD43 in a cell population by flow cytometry, and to identify the “CD43−” or “CD43+” phenotype. In another example, the “CD34+” phenotype may be identified by using an antibody specific for CD34 conjugated to the phycoerythrin-cyanine 7 (PE-Cy7) complex, which will bind to cells bearing the CD34 marker, and which can be quantified by flow cytometry. CD34, also called “gp105-120”, is a transmembrane phosphoglycoprotein of about 105 to 120 kD, belonging to the sialomucin family.

“Therapy,” “therapeutic,” “treating,” “treat” or “treatment”, as used herein, refers broadly to treating a disease, arresting or reducing the development of the disease or its clinical symptoms, and/or relieving the disease, causing regression of the disease or its clinical symptoms. “Therapy”, “therapeutic,” “treating,” “treat” or “treatment” encompasses prophylaxis, prevention, treatment, cure, remedy, reduction, alleviation, and/or providing relief from a disease, signs, and/or symptoms of a disease. “Therapy”, “therapeutic,” “treating,” “treat” or “treatment” encompasses an alleviation of signs and/or symptoms in patients with ongoing disease signs and/or symptoms (e.g., muscle weakness, multiple sclerosis.) “Therapy”, “therapeutic,” “treating,” “treat” or “treatment” also encompasses “prophylaxis” and “prevention”. Prophylaxis includes preventing disease occurring subsequent to treatment of a disease in a patient or reducing the incidence or severity of the disease in a patient. The term “reduced”, for purpose of therapy, “therapeutic,” “treating,” “treat” or “treatment” refers broadly to the clinical significant reduction in signs and/or symptoms. “Therapy”, “therapeutic,” “treating,” “treat” or “treatment” includes treating relapses or recurrent signs and/or symptoms (e.g., retinal degeneration, loss of vision.) “Therapy”, “therapeutic,” “treating,” “treat” or “treatment” encompasses but is not limited to precluding the appearance of signs and/or symptoms anytime as well as reducing existing signs and/or symptoms and eliminating existing signs and/or symptoms. “Therapy”, “therapeutic,” “treating,” “treat” or “treatment” includes treating chronic disease (“maintenance”) and acute disease. For example, treatment includes treating or preventing relapses or the recurrence of signs and/or symptoms (e.g., muscle weakness, multiple sclerosis).

Further exemplary pluripotent stem cells include induced pluripotent stem cells (iPS cells) generated by reprogramming a somatic cell by expressing or inducing expression of a combination of factors (“reprogramming factors”). iPS cells may be generated using fetal, postnatal, newborn, juvenile, or adult somatic cells. iPS cells may be obtained from a cell bank. Alternatively, iPS cells may be newly generated (by processes known in the art) prior to commencing differentiation to RPE cells or another cell type. The making of iPS cells may be an initial step in the production of differentiated cells. iPS cells may be specifically generated using material from a particular patient or matched donor with the goal of generating tissue-matched RPE cells. iPS cells can be produced from cells that are not substantially immunogenic in an intended recipient, e.g., produced from autologous cells or from cells histocompatible to an intended recipient. As further discussed above (see “pluripotent cells”), pluripotent cells including iPS cells may be genetically modified or otherwise modified to increase longevity, potency, homing, or to deliver a desired factor in cells that are differentiated from such pluripotent cells (for example, MSCs and hemangioblasts).

As a further example, induced pluripotent stem cells may be generated by reprogramming a somatic or other cell by contacting the cell with one or more reprogramming factors. For example, the reprogramming factor(s) may be expressed by the cell, e.g., from an exogenous nucleic acid added to the cell, or from an endogenous gene in response to a factor such as a small molecule, microRNA, or the like that promotes or induces expression of that gene (see Suh and Blelloch, Development 138, 1653-1661 (2011); Miyosh et al., Cell Stem Cell (2011), doi:10.1016/j.stem.2011.05.001; Sancho-Martinez et al., Journal of Molecular Cell Biology (2011) 1-3; Anokye-Danso et al., Cell Stem Cell 8, 376-388, Apr. 8, 2011; Orkin and Hochedlinger, Cell 145, 835-850, Jun. 10, 2011, each of which is incorporated by reference herein in its entirety). Reprogramming factors may be provided from an exogenous source, e.g., by being added to the culture media, and may be introduced into cells by methods known in the art such as through coupling to cell entry peptides, protein or nucleic acid transfection agents, lipofection, electroporation, biolistic particle delivery system (gene gun), microinjection, and the like. iPS cells can be generated using fetal, postnatal, newborn, juvenile, or adult somatic cells. In certain embodiments, factors that can be used to reprogram somatic cells to pluripotent stem cells include, for example, a combination of Oct4 (sometimes referred to as Oct 3/4), Sox2, c-Myc, and Klf4. In other embodiments, factors that can be used to reprogram somatic cells to pluripotent stem cells include, for example, a combination of Oct-4, Sox2, Nanog, and Lin28. In other embodiments, somatic cells are reprogrammed by expressing at least 2 reprogramming factors, at least three reprogramming factors, or four reprogramming factors. In other embodiments, additional reprogramming factors are identified and used alone or in combination with one or more known reprogramming factors to reprogram a somatic cell to a pluripotent stem cell. iPS cells typically can be identified by expression of the same markers as embryonic stem cells, though a particular iPS cell line may vary in its expression profile.

The induced pluripotent stem cell may be produced by expressing or inducing the expression of one or more reprogramming factors in a somatic cell. The somatic cell is a fibroblast, such as a dermal fibroblast, synovial fibroblast, or lung fibroblast, or a non-fibroblastic somatic cell. The somatic cell is also a mesenchymal stem cell. The somatic cell is reprogrammed by expressing at least 1, 2, 3, 4, 5 reprogramming factors. The reprogramming factors may be selected from Oct 3/4, Sox2, NANOG, Lin28, c Myc, and Klf4. Expression of the reprogramming factors may be induced by contacting the somatic cells with at least one agent, such as a small organic molecule agent, that induces expression of reprogramming factors.

The somatic cell may also be reprogrammed using a combinatorial approach wherein the reprogramming factor is expressed (e.g., using a viral vector, plasmid, and the like) and the expression of the reprogramming factor is induced (e.g., using a small organic molecule.) For example, reprogramming factors may be expressed in the somatic cell by infection using a viral vector, such as a retroviral vector or a lentiviral vector. Also, reprogramming factors may be expressed in the somatic cell using a non-integrative vector, such as an episomal plasmid. See, e.g., Yu et al., Science. 2009 May 8; 324(5928):797-801, which is hereby incorporated by reference in its entirety. When reprogramming factors are expressed using non-integrative vectors, the factors may be expressed in the cells using electroporation, transfection, or transformation of the somatic cells with the vectors. For example, in mouse cells, expression of four factors (Oct3/4, Sox2, c myc, and Klf4) using integrative viral vectors can be used to reprogram a somatic cell. In human cells, expression of four factors (Oct34, Sox2, NANOG, and Lin28) using integrative viral vectors can be used to reprogram a somatic cell.

Once the reprogramming factors are expressed in the cells, the cells may be cultured. Over time, cells with ES characteristics appear in the culture dish. The cells may be chosen and subcultured based on, for example, ES morphology, or based on expression of a selectable or detectable marker. The cells may be cultured to produce a culture of cells that resemble ES cells—these are putative iPS cells. iPS cells typically can be identified by expression of the same markers as other embryonic stem cells, though a particular iPS cell line may vary in its expression profile. Exemplary iPS cells may express Oct-4, alkaline phosphatase, SSEA 3 surface antigen, SSEA 4 surface antigen, TRA 1 60, anchor TRA 1 81.

The invention provides for generation of regenerative T cells through differentiation of pluripotent stem cells in the presence of mesenchymal stem cells or products derived thereof. Said products include exosomes, microvesicles and apoptotic bodies.

According to a preferred embodiment, the pluripotent stem cells are cultured under conditions enabling to induce the formation of embryoid bodies. For this purpose, cell culture can be performed, for illustrative purposes, in a low-adhesion plate (Sigma-Aldrich, Fisher), which favors the appearance of cell aggregates in three dimensions (embryoid bodies) and reproduces in a more efficient way the intercellular interactions existing during the development of the embryo in the body of the animal.

In an embodiment, the culture of pluripotent stem cells is performed in a culture medium suitable to induce the formation of embryoid bodies, for at least 9 days, under conditions allowing to obtain embryoid bodies comprising at least 5% of CD34+CD43+ cells. The invention teaches that this step may be performed in the presence of interferon gamma activated mesenchymal stem cells, preferably at a ratio of one to one. Culture media suitable for the growth of hematopoietic cells are known from the state of the art. According to an embodiment, the culture medium to induce the formation of embryoid bodies comprises a serum-free culture medium suitable for the growth of hematopoietic cells, for example the StemPro-34 SFM medium (ThermoFisher). According to an embodiment, the culture medium to induce the formation of embryoid bodies comprises from about 0.1 to about 5% of L-glutamine, preferably about 1% of L-glutamine. According to an embodiment, the culture medium to induce the formation of embryoid bodies comprises from about 0.1% to about 5% of non-essential amino acids, preferably about 1% of non-essential amino acids. According to an embodiment, the culture medium to induce the formation of embryoid bodies comprises from about 0.01% to about 0.5% of 2-mercaptoethanol, preferably about 0.1% of 2-mercaptoethanol. According to an embodiment, the culture medium to induce the formation of embryoid bodies comprises from about 10 to about 1000 U/mL of penicillin, preferably about 100 U/mL of penicillin. According to an embodiment, the culture medium to induce the formation of embryoid bodies comprises from about 10 to about 1000 ng/mL of streptomycin, preferably about 100 ng/mL of streptomycin. According to an embodiment, the culture medium to induce the formation of embryoid bodies comprises from about 5 to about 1000 .mu.g/mL, preferably about 50 .mu.g/mL of ascorbic acid. According to an embodiment, a serum-free culture medium suitable for the growth of hematopoietic cells, for example StemPro-34 SFM medium, comprises L-glutamine, non-essential amino acids, 2-mercaptoethanol, penicillin, streptomycin and/or ascorbic acid as described above. In an embodiment, the culture of the pluripotent stem cells in step a) is carried out in a serum-free culture medium suitable for the growth of hematopoietic cells, for example StemPro-34 SFM medium (ThermoFisher), comprising BMP, FGF2, VEGF, SCF, Flt3-L and/or IL-3, for at least 9 days, under conditions allowing to obtain embryoid bodies comprising at least 5% of CD34+CD43+ cells. In an embodiment, the culture of the pluripotent stem cells in step a) is carried out in a serum-free culture medium suitable for the growth of hematopoietic cells, for example StemPro-34 SFM medium (ThermoFisher), comprising BMP, FGF2, VEGF, SCF, Flt3-L and IL-3, for at least 9 days, under conditions allowing to obtain embryoid bodies comprising at least 5% of CD34+CD43+ cells. According to a preferred embodiment, the pluripotent stem cells are first incubated in the presence of BMP (bone morphogenetic protein) to facilitate the induction of the formation of embryoid bodies. According to a preferred embodiment, the pluripotent stem cells are incubated for 10 to 48 hours, preferably for one day, in the presence of BMP. According to another embodiment, the cells are incubated in the presence of 3 to 300 ng/mL of BMP, preferably from 10 to 100 ng/mL of BMP, more preferably from 20 to 50 ng/mL of BMP, and particularly about 30 ng/mL of BMP. Preferably, the BMP is BMP-4, more preferably human BMP-4 (hBMP-4). More preferably, the cells are incubated for one day in the presence of about 30 ng/mL of hBMP-4a, preferably on DO (start of the second phase). In an embodiment, after the incubation in the presence of BMP, preferably BMP-4, a mixture comprising BMP (preferably BMP-4) and FGF-2 is added to the medium to allow induction of mesoderm. Preferably between about 3 ng/mL and about 300 ng/mL of BMP, preferably BMP-4, and between about 0.5 ng/mL and about 50 ng/mL of FGF2 is added to the medium, preferably about 30 ng/mL of BMP, preferably BMP-4, and about 5 ng/mL of FGF2 are added to the medium. According to an embodiment, this addition is carried out all at once, preferably on day 1 of the phase of induction of the formation of embryoid bodies (day D1). In an embodiment, a solution comprising growth factors and/or cytokines is added every two days, preferably from day 3 of the phase of induction of the formation of embryoid bodies (day D3), until the end of the induction phase of the formation of embryoid bodies. The end of the induction phase of the formation of embryoid bodies corresponds to the moment when the embryoid bodies are dissociated. This can for example occur on day 5, day 6, day 7, day 8, day 9, day 10, day 11, day 12 of the induction phase of the formation of the embryoid bodies, or later (D5, D6, D7, D8, D9, D10, D11, D12 or later). In a particular embodiment, step a) of culture of the pluripotent stem cells is carried out for at least 9 days. In an embodiment, step a) is carried out for 9 to 17 days, preferably for 9 to 15 days, preferably for 9 to 14 days. In an embodiment, step a) is carried out for 9 to 12 days, preferably for 9 to 11 days, preferably for 9 to 10 days. In an embodiment, the solution comprising growth factors and/or cytokines comprises VEGF (vascular endothelial growth factor, ThermoFisher), SCF (stem cell growth factor, ThermoFisher), FLt3-L (Fms-like tyrosine kinase 3-ligand, ThermoFisher), IL-3 (recombinant interleukin 3, ThermoFisher) and/or FGF2.

A preferred solution comprising growth factors and/or cytokines comprises VEGF (vascular endothelial growth factor, ThermoFisher), SCF (stem cell growth factor, ThermoFisher), FLt3-L (Fms-like tyrosine kinase 3-ligand, ThermoFisher), IL-3 (recombinant interleukin 3 ThermoFisher) and FGF2. Preferably, the solution comprising growth factors and/or cytokines comprises from about 2 ng/mL to about 200 ng/mL VEGF, preferably about 20 ng/mL. Preferably, the solution comprising growth factors and/or cytokines comprises from about 10 ng/mL to about 300 ng/mL of SCF, preferably about 100 ng/mL of SCF. Preferably, the solution comprising growth factors and/or cytokines comprises from about 2 ng/mL to about 200 ng/mL of Flt3L, preferably about 20 ng/mL of Flt3L. Preferably, a solution comprising growth factors and/or cytokines which does not comprise FGF2 is used just before the end of the phase of induction of the formation of embryoid bodies. Preferably, this solution is used from day D7 during the phase of induction of the formation of embryoid bodies. According to an embodiment of the invention, the embryoid bodies obtained comprise hematopoietic stem cells capable of expressing the CD34 marker. Thus, the inventors have shown that the first step of the method of the invention allows to obtain a subpopulation of hematopoietic stem cells exhibiting the CD34+ phenotype. More particularly, about 40% of the total cell population expresses CD34+ after 7 days of culture (first phase). The inventors have further demonstrated the obtaining of a CD34+CD43+ subpopulation and a CD34+CD43− subpopulation, these two populations being present in relatively equivalent proportions in the population. Surprisingly, the inventors have shown that the increase of the CD34+CD43+ subpopulation in the embryoid bodies, and the presence of the CD34+CD43− subpopulation in the embryoid bodies, make the total cell population more suitable for continuing with the cell differentiation protocol according to the invention.

For the practice of the invention, MSC are a type of stem cell utilized for inducing regeneration of the endometrium. “Mesenchymal stem cell” or “MSC” in some embodiments refers to cells that are (1) adherent to plastic, (2) express CD73, CD90, and CD105 antigens, while being CD14, CD34, CD45, and HLA-DR negative, and (3) possess ability to differentiate to osteogenic, chondrogenic and adipogenic lineage. Other cells possessing mesenchymal-like properties are included within the definition of “mesenchymal stem cell”, with the condition that said cells possess at least one of the following: a) regenerative activity; b) production of growth factors; c) ability to induce a healing response, either directly, or through elicitation of endogenous host repair mechanisms. As used herein, “mesenchymal stromal cell” or mesenchymal stem cell can be used interchangeably. Said MSC can be derived from any tissue including, but not limited to, bone marrow, adipose tissue, amniotic fluid, endometrium, trophoblast-derived tissues, cord blood, Wharton jelly/umbilical cord tissue, placenta, amniotic tissue, derived from pluripotent stem cells, and tooth. In some definitions of “MSC”, said cells include cells that are CD34 positive upon initial isolation from tissue but are similar to cells described about phenotypically and functionally. As used herein, “MSC” may include cells that are isolated from tissues using cell surface markers selected from the list comprised of NGF-R, PDGF-R, EGF-R, IGF-R, CD29, CD49a, CD56, CD63, CD73, CD105, CD106, CD140b, CD146, CD271, MSCA-1, SSEA4, STRO-1 and STRO-3 or any combination thereof, and satisfy the ISCT criteria either before or after expansion. Furthermore, as used herein, in some contexts, “MSC” includes cells described in the literature as bone marrow stromal stem cells (BMSSC), marrow-isolated adult multipotent inducible cells (MIAMI) cells, multipotent adult progenitor cells (MAPC), mesenchymal adult stem cells (MASCS), MultiStem®, Prochymal®, remestemcel-L, Mesenchymal Precursor Cells (MPCs), Dental Pulp Stem Cells (DPSCs), PLX cells, PLX-PAD, AlloStem®, Astrostem®, Ixmyelocel-T, MSC-NTF, NurOwn™ Stemedyne™-MSC, Stempeucel®, StempeucelCLI, StempeucelOA, HiQCell, Hearticellgram-AMI, Revascor®, Cardiorel®, Cartistem®, Pneumostem®, Promostem®, Homeo-GH, AC607, PDA001, SB623, CX601, AC607, Endometrial Regenerative Cells (ERC), adipose-derived stem and regenerative cells (ADRCs).

In one embodiment, the cells of the present invention are generally referred to as umbilical-derived cells (or UDCs). They also may sometimes be referred to more generally herein as postpartum-derived cells or postpartum cells (PPDCs). In addition, the cells may be described as being stem or progenitor cells, the latter term being used in the broad sense. The term derived is used to indicate that the cells have been obtained from their biological source and grown or otherwise manipulated in vitro (e.g., cultured in a growth medium to expand the population and/or to produce a cell line). The in vitro manipulations of umbilical stem cells and the unique features of the umbilicus-derived cells of the present invention are described in detail below.

Various terms are used to describe cells in culture. Cell culture refers generally to cells taken from a living organism and grown under controlled condition (“in culture” or “cultured”). A primary cell culture is a culture of cells, tissues, or organs taken directly from an organism(s) before the first subculture. Cells are expanded in culture when they are placed in a growth medium under conditions that facilitate cell growth and/or division, resulting in a larger population of the cells. When cells are expanded in culture, the rate of cell proliferation is sometimes measured by the amount of time needed for the cells to double in number. This is referred to as doubling time.

A conditioned medium is a medium in which a specific cell or population of cells has been cultured, and then removed. When cells are cultured in a medium, they may secrete cellular factors that can provide trophic support to other cells. Such trophic factors include, but are not limited to hormones, cytokines, extracellular matrix (ECM), proteins, vesicles, antibodies, and granules. The medium containing the cellular factors is the conditioned medium. In one specific embodiment of the invention, supernatant is collected from MSC selected for ability to suppress fibrosis. In other embodiments, MSC are chosen based on angiogenic activity. Said angiogenic activity is identified based on proteomic and other analysis of markers, proteins, and peptides that are correlated with enhanced ability to induce regeneration. In a specific embodiment the invention provides means of regenerating endometrium using said conditioned media. In some embodiments of the invention, the inventors interchangeably use the words “conditioned media” and “trophic factors”. Generally, a trophic factor is defined as a substance that promotes or at least supports, survival, growth, proliferation and/or maturation of a cell, or stimulates increased activity of a cell.

When referring to cultured vertebrate cells, the term senescence (also replicative senescence or cellular senescence) refers to a property attributable to finite cell cultures; namely, their inability to grow beyond a finite number of population doublings (sometimes referred to as Hayflick's limit). Although cellular senescence was first described using fibroblast-like cells, most normal human cell types that can be grown successfully in culture undergo cellular senescence. The in vitro lifespan of different cell types varies, but the maximum lifespan is typically fewer than 100 population doublings (this is the number of doublings for all the cells in the culture to become senescent and thus render the culture unable to divide). Senescence does not depend on chronological time, but rather is measured by the number of cell divisions, or population doublings, the culture has undergone. Thus, cells made quiescent by removing essential growth factors are able to resume growth and division when the growth factors are re-introduced, and thereafter carry out the same number of doublings as equivalent cells grown, continuously. Similarly, when cells are frozen in liquid nitrogen after various numbers of population doublings and then thawed and cultured, they undergo substantially the same number of doublings as cells maintained unfrozen in culture. Senescent cells are not dead or dying cells; they are actually resistant to programmed cell death (apoptosis), and have been maintained in their nondividing state for as long as three years. These cells are very much alive and metabolically active, but they do not divide. The nondividing state of senescent cells has not yet been found to be reversible by any biological, chemical, or viral agent.

As used herein, the term Growth Medium generally refers to a medium sufficient for the culturing of umbilicus-derived cells. In particular, one presently preferred medium for the culturing of the cells of the invention herein comprises Dulbecco's Modified Essential Media (also abbreviated DMEM herein). Particularly preferred is DMEM-low glucose (also DMEM-LG herein) (Invitrogen, Carlsbad, Calif.). The DMEM-low glucose is preferably supplemented with 15% (v/v) fetal bovine serum (e.g. defined fetal bovine serum, Hyclone, Logan Utah), antibiotics/antimycotics (preferably penicillin (100 Units/milliliter), streptomycin (100 milligrams/milliliter), and amphotericin B (0.25 micrograms/milliliter), (Invitrogen, Carlsbad, Calif.)), and 0.001% (v/v) 2-mercaptoethanol (Sigma, St. Louis Mo.). In some cases, different growth media are used, or different supplementations are provided, and these are normally indicated in the text as supplementations to Growth Medium. Growth Medium can also include xeno-free defined components and can also be lyophilized platelet lysate or PRP.

Also relating to the present invention, the term standard growth conditions, as used herein refers to culturing of cells at 37.degree. C., in a standard atmosphere comprising 5% CO.sub.2. Relative humidity is maintained at about 100%. While foregoing the conditions are useful for culturing, it is to be understood that such conditions are capable of being varied by the skilled artisan who will appreciate the options available in the art for culturing cells, for example, varying the temperature, CO.sub.2, relative humidity, oxygen, growth medium, and the like.

Oct-4 (oct-3 in humans) is a transcription factor expressed in the pregastrulation embryo, early cleavage stage embryo, cells of the inner cell mass of the blastocyst, and embryonic carcinoma (“EC”) cells (Nichols, J. et al. (1998) Cell 95: 379-91), and is down-regulated when cells are induced to differentiate. The oct-4 gene (oct-3 in humans) is transcribed into at least two splice variants in humans, oct-3A and oct-3B. The oct-3B splice variant is found in many differentiated cells whereas the oct-3A splice variant (also previously designated oct-3/4) is reported to be specific for the undifferentiated embryonic stem cell. See Shimozaki et al. (2003) Development 130: 2505-12. Expression of oct-3/4 plays an important role in determining early steps in embryogenesis and differentiation. Oct-3/4, in combination with rox-1, causes transcriptional activation of the Zn-finger protein rex-1, which is also required for maintaining ES cells in an undifferentiated state (Rosfjord, E. and Rizzino, A. (1997) Biochem Biophys Res Commun 203: 1795-802; Ben-Shushan, E. et al. (1998) Mol Cell Biol 18: 1866-78).

In one embodiment MSC donor lots are generated from umbilical cord tissue. Means of generating umbilical cord tissue MSC have been previously published and are incorporated by reference [1-7]. The term “umbilical tissue derived cells (UTC)” refers, for example, to cells as described in U.S. Pat. Nos. 7,510,873, 7,413,734, 7,524,489, and 7,560,276. The UTC can be of any mammalian origin e.g. human, rat, primate, porcine and the like. In one embodiment of the invention, the UTC are derived from human umbilicus. Umbilicus-derived cells, which relative to a human cell that is a fibroblast, a mesenchymal stem cell, or an iliac crest bone marrow cell, have reduced expression of genes for one or more of: short stature homeobox 2; heat shock 27 kDa protein 2; chemokine (C-X-C motif) ligand 12 (stromal cell-derived factor 1); elastin (supravalvular aortic stenosis, Williams-Beuren syndrome); Homo sapiens mRNA; cDNA DKFZp586M2022 (from clone DKFZp586M2022); mesenchyme homeobox 2 (growth arrest-specific homeobox); sine oculis homeobox homolog 1 (Drosophila); crystallin, alpha B; disheveled associated activator of morphogenesis 2; DKFZP586B2420 protein; similar to neuralin 1; tetranectin (plasminogen binding protein); src homology three (SH3) and cysteine rich domain; cholesterol 25-hydroxylase; runt-related transcription factor 3; interleukin 11 receptor, alpha; procollagen C-endopeptidase enhancer; frizzled homolog 7 (Drosophila); hypothetical gene BC008967; collagen, type VIII, alpha 1; tenascin C (hexabrachion); iroquois homeobox protein 5; hephaestin; integrin, beta 8; synaptic vesicle glycoprotein 2; neuroblastoma, suppression of tumorigenicity 1; insulin-like growth factor binding protein 2, 36 kDa; Homo sapiens cDNA FLJ12280 fis, clone MAMMA1001744; cytokine receptor-like factor 1; potassium intermediate/small conductance calcium-activated channel, subfamily N, member 4; integrin, beta 7; transcriptional co-activator with PDZ-binding motif (TAZ); sine oculis homeobox homolog 2 (Drosophila); KIAA1034 protein; vesicle-associated membrane protein 5 (myobrevin); EGF-containing fibulin-like extracellular matrix protein 1; early growth response 3; distal-less homeobox 5; hypothetical protein FLJ20373; aldo-keto reductase family 1, member C3 (3-alpha hydroxysteroid dehydrogenase, type II); biglycan; transcriptional co-activator with PDZ-binding motif (TAZ); fibronectin 1; proenkephalin; integrin, beta-like 1 (with EGF-like repeat domains); Homo sapiens mRNA full length insert cDNA clone EUROIMAGE 1968422; EphA3; KIAA0367 protein; natriuretic peptide receptor C/guanylate cyclase C (atrionatriuretic peptide receptor C); hypothetical protein FLJ14054; Homo sapiens mRNA; cDNA DKFZp564B222 (from clone DKFZp564B222); BCL2/adenovirus E1B 19 kDa interacting protein 3-like; AE binding protein 1; and cytochrome c oxidase subunit VIIa polypeptide 1 (muscle). In addition, these isolated human umbilicus-derived cells express a gene for each of interleukin 8; reticulon 1; chemokine (C-X-C motif) ligand 1 (melonoma growth stimulating activity, alpha); chemokine (C-X-C motif) ligand 6 (granulocyte chemotactic protein 2); chemokine (C-X-C motif) ligand 3; and tumor necrosis factor, alpha-induced protein 3, wherein the expression is increased relative to that of a human cell which is a fibroblast, a mesenchymal stem cell, an iliac crest bone marrow cell, or placenta-derived cell. The cells are capable of self-renewal and expansion in culture, and have the potential to differentiate into cells of other phenotypes.

Methods of deriving cord tissue mesenchymal stem cells from human umbilical tissue are provided. The cells are capable of self-renewal and expansion in culture, and have the potential to differentiate into cells of other phenotypes. The method comprises (a) obtaining human umbilical tissue; (b) removing substantially all of blood to yield a substantially blood-free umbilical tissue, (c) dissociating the tissue by mechanical or enzymatic treatment, or both, (d) resuspending the tissue in a culture medium, and (e) providing growth conditions which allow for the growth of a human umbilicus-derived cell capable of self-renewal and expansion in culture and having the potential to differentiate into cells of other phenotypes. Tissue can be obtained from any completed pregnancy, term or less than term, whether delivered vaginally, or through other routes, for example surgical Cesarean section. Obtaining tissue from tissue banks is also considered within the scope of the present invention.

The tissue is rendered substantially free of blood by any means known in the art. For example, the blood can be physically removed by washing, rinsing, and diluting and the like, before or after bulk blood removal for example by suctioning or draining. Other means of obtaining a tissue substantially free of blood cells might include enzymatic or chemical treatment.

Dissociation of the umbilical tissues can be accomplished by any of the various techniques known in the art, including by mechanical disruption, for example, tissue can be aseptically cut with scissors, or a scalpel, or such tissue can be otherwise minced, blended, ground, or homogenized in any manner that is compatible with recovering intact or viable cells from human tissue.

In a presently preferred embodiment, the isolation procedure also utilizes an enzymatic digestion process. Many enzymes are known in the art to be useful for the isolation of individual cells from complex tissue matrices to facilitate growth in culture. As discussed above, a broad range of digestive enzymes for use in cell isolation from tissue is available to the skilled artisan. Ranging from weakly digestive (e.g. deoxyribonucleases and the neutral protease, dispase) to strongly digestive (e.g. papain and trypsin), such enzymes are available commercially. A non-exhaustive list of enzymes compatible herewith includes mucolytic enzyme activities, metalloproteases, neutral proteases, serine proteases (such as trypsin, chymotrypsin, or elastase), and deoxyribonucleases. Presently preferred are enzyme activities selected from metalloproteases, neutral proteases and mucolytic activities. For example, collagenases are known to be useful for isolating various cells from tissues. Deoxyribonucleases can digest single-stranded DNA and can minimize cell-clumping during isolation. Enzymes can be used alone or in combination. Serine protease are preferably used in a sequence following the use of other enzymes as they may degrade the other enzymes being used. The temperature and time of contact with serine proteases must be monitored. Serine proteases may be inhibited with alpha 2 microglobulin in serum and therefore the medium used for digestion is preferably serum-free. EDTA and DNase are commonly used and may improve yields or efficiencies. Preferred methods involve enzymatic treatment with for example collagenase and dispase, or collagenase, dispase, and hyaluronidase, and such methods are provided wherein in certain preferred embodiments, a mixture of collagenase and the neutral protease dispase are used in the dissociating step. More preferred are those methods which employ digestion in the presence of at least one collagenase from Clostridium histolyticum, and either of the protease activities, dispase and thermolysin. Still more preferred are methods employing digestion with both collagenase and dispase enzyme activities. Also preferred are methods which include digestion with a hyaluronidase activity in addition to collagenase and dispase activities. The skilled artisan will appreciate that many such enzyme treatments are known in the art for isolating cells from various tissue sources. For example, the LIBERASE BLENDZYME (Roche) series of enzyme combinations of collagenase and neutral protease are very useful and may be used in the instant methods. Other sources of enzymes are known, and the skilled artisan may also obtain such enzymes directly from their natural sources. The skilled artisan is also well-equipped to assess new, or additional enzymes or enzyme combinations for their utility in isolating the cells of the invention. Preferred enzyme treatments are 0.5, 1, 1.5, or 2 hours long or longer. In other preferred embodiments, the tissue is incubated at 37.degree. C. during the enzyme treatment of the dissociation step. Diluting the digest may also improve yields of cells as cells may be trapped within a viscous digest. A version of the cell can also with made with non-enzymatic components such as TryplE.

While the use of enzyme activities is presently preferred, it is not required for isolation methods as provided herein. Methods based on mechanical separation alone may be successful in isolating the instant cells from the umbilicus as discussed above.

The cells can be resuspended after the tissue is dissociated into any culture medium as discussed herein above. Cells may be resuspended following a centrifugation step to separate out the cells from tissue or other debris. Resuspension may involve mechanical methods of resuspending, or simply the addition of culture medium to the cells.

Providing the growth conditions allows for a wide range of options as to culture medium, supplements, atmospheric conditions, and relative humidity for the cells. A preferred temperature is 37.degree. C., however the temperature may range from about 35.degree. C. to 39.degree. C. depending on the other culture conditions and desired use of the cells or culture.

Presently preferred are methods which provide cells which require no exogenous growth factors, except as are available in the supplemental serum provided with the Growth Medium. Also provided herein are methods of deriving umbilical cells capable of expansion in the absence of particular growth factors. The methods are similar to the method above, however they require that the particular growth factors (for which the cells have no requirement) be absent in the culture medium in which the cells are ultimately resuspended and grown in. In this sense, the method is selective for those cells capable of division in the absence of the particular growth factors. Preferred cells in some embodiments are capable of growth and expansion in chemically-defined growth media with no serum added. In such cases, the cells may require certain growth factors, which can be added to the medium to support and sustain the cells. Presently preferred factors to be added for growth on serum-free media include one or more of FGF, EGF, IGF, and PDGF. In more preferred embodiments, two, three or all four of the factors are add to serum free or chemically defined media. In other embodiments, LIF is added to serum-free medium to support or improve growth of the cells.

Also provided are methods wherein the cells can expand in the presence of from about 5% to about 20% oxygen in their atmosphere. Methods to obtain cells that require L-valine require that cells be cultured in the presence of L-valine. After a cell is obtained, its need for L-valine can be tested and confirmed by growing on D-valine containing medium that lacks the L-isomer.

Methods are provided wherein the cells can undergo at least 25, 30, 35, or 40 doublings prior to reaching a senescent state. Methods for deriving cells capable of doubling to reach 10.sup.14 cells or more are provided. Preferred are those methods which derive cells that can double sufficiently to produce at least about 10.sup.14, 10.sup.15, 10.sup.16, or 10.sup.17 or more cells when seeded at from about 10.sup.3 to about 10.sup.6 cells/cm.sup.2 in culture. Preferably these cell numbers are produced within 80, 70, or 60 days or less. In one embodiment, cord tissue mesenchymal stem cells are isolated and expanded, and possess one or more markers selected from a group comprising of CD10, CD13, CD44, CD73, CD90, CD141, PDGFr-alpha, or HLA-A, B, C. In addition, the cells do not produce one or more of CD31, CD34, CD45, CD117, CD141, or HLA-DR, DP, DQ.

Exosomes, also referred to as “particles” may comprise vesicles or a flattened sphere limited by a lipid bilayer. The particles may comprise diameters of 40-100 nm. The particles may be formed by inward budding of the endosomal membrane. The particles may have a density of about 1.13-1.19 g/ml and may float on sucrose gradients. The particles may be enriched in cholesterol and sphingomyelin, and lipid raft markers such as GM1, GM3, flotillin and the src protein kinase Lyn. The particles may comprise one or more proteins present in mesenchymal stem cell or mesenchymal stem cell conditioned medium such as a protein characteristic or specific to the MESENCHYMALSTEM CELL or MESENCHYMALSTEM CELL-CM. They may comprise RNA, for example miRNA. Said particles may possess one or more genes or gene products found in Mesenchymal stem cell or medium which is conditioned by culture of Mesenchymal stem cell. The particle may comprise molecules secreted by the MESENCHYMALSTEM CELL. Such a particle, and combinations of any of the molecules comprised therein, including in particular proteins or polypeptides, may be used to supplement the activity of, or in place of, the Mesenchymal stem cell or medium conditioned by the Mesenchymal stem cell for the purpose of for example treating or preventing a disease. Said particle may comprise a cytosolic protein found in cytoskeleton e.g. tubulin, actin and actin-binding proteins, intracellular membrane fusions and transport e.g. annexins and rab proteins, signal transduction proteins e.g. protein kinases, 14-3-3 and heterotrimeric G proteins, metabolic enzymes e.g. peroxidases, pyruvate and lipid kinases, and enolase-1 and the family of tetraspanins e.g. CD9, CD63, CD81 and CD82. In particular, the particle may comprise one or more tetraspanins. The particles may comprise mRNA and/or microRNA. In one embodiment, mesenchymalstem cell exosomes, or particles may be produced by culturing mesenchymal stem cells in a medium to condition it. The mesenchymal stem cells may comprise human umbilical tissue derived cells which possess markers selected from a group comprising of CD90, CD73 and CD105. The medium may comprise DMEM. The DMEM may be such that it does not comprise phenol red. The medium may be supplemented with insulin, transferrin, or selenoprotein (ITS), or any combination thereof. It may comprise FGF2. It may comprise PDGF AB. The concentration of FGF2 may be about 5 ng/ml FGF2. The concentration of PDGF AB may be about 5 ng/ml. The medium may comprise glutamine-penicillin-streptomycin or b-mercaptoethanol, or any combination thereof. The cells may be cultured for about 1, 2, 3, 4, 5, 6, 7, 8, 9, 10 days or more, for example 3 days. The conditioned medium may be obtained by separating the cells from the medium. The conditioned medium may be centrifuged, for example at 500 g. it may be concentrated by filtration through a membrane. The membrane may comprise a >1000 kDa membrane. The conditioned medium may be concentrated about 50 times or more. The conditioned medium may be subject to liquid chromatography such as HPLC. The conditioned medium may be separated by size exclusion. Any size exclusion matrix such as Sepharose may be used. As an example, a TSK Guard column SWXL, 6.times.40 mm or a TSK gel G4000 SWXL, 7.8.times.300 mm may be employed. The eluent buffer may comprise any physiological medium such as saline. It may comprise 20 mM phosphate buffer with 150 mM of NaCl at pH 7.2. The chromatography system may be equilibrated at a flow rate of 0.5 ml/min. The elution mode may be isocratic. UV absorbance at 220 nm may be used to track the progress of elution. Fractions may be examined for dynamic light scattering (DLS) using a quasi-elastic light scattering (QELS) detector. Fractions which are found to exhibit dynamic light scattering may be retained. For example, a fraction which is produced by the general method as described above, and which elutes with a retention time of 11-13 minutes, such as 12 minutes, is found to exhibit dynamic light scattering. The r.sub.h of particles in this peak is about 45-55 nm. Such fractions comprise mesenchymal stem cell particles such as exosomes. The final cell product has karyotype stability to at least 50 passages.

1. A method for production of T cells possessing ability to regenerate injured tissue comprising the steps of: a) obtaining a pluripotent stem cell; b) differentiating said pluripotent stem cell into a T cell; c) contacting said pluripotent stem cell at one or more time points during the differentiation process with mesenchymal stem cells or products derived thereof; and d) isolating T cells possessing regenerative activity.

2. The method of claim 1, wherein said pluripotent stem cells are selected from the group consisting of: a) inducible pluripotent stem cells; b) somatic cell nuclear transfer derived stem cells; c) embryonic stem cells; and d) parthenogenic derived stem cells.

3. The method of claim 1, wherein said pluripotent stem cells are differentiated into T cells by sequential culture in cytokines and conditions replicating thymopoiesis.

4. The method of claim 3, wherein said culture conditions comprise of: a) de-aggregating pluripotent stem cells; b) treating said cells with interleukin-7 for 1-7 days at a concentration of 0.1-100 pg/ml; c) subsequently treating said cells with interleukin-2 and interleukin-7 for an additional 1-14 days; d) optionally contacting the cells in step “c” with agonistic antibody to CD3 at a sufficient concentration to induce phosphorylation of TCR zeta chain; and e) extracting non-adherent cells from the culture.

5. The method of claim 2, wherein said embryonic stem cell population expresses genes selected from the group consisting of: stage-specific embryonic antigens (SSEA) 3, SSEA 4, Tra-1-60 and Tra-1-81, Oct-3/4, Cripto, gastrin-releasing peptide (GRP) receptor, podocalyxin-like protein (PODXL), Rex-1, GCTM-2, Nanog, and human telomerase reverse transcriptase (hTERT).

6. The method of claim 2, wherein said inducible pluripotent stem cell possesses markers selected from the group consisting of: CD10, CD13, CD44, CD73, CD90, PDGFr-alpha, PD-L2, and HLA-A, -B, -C and possesses ability to undergo at least 40 doublings in culture, while maintaining a normal karyotype upon passaging up to 50 times.

7. The method of claim 2, wherein said somatic cell nuclear transfer derived stem cells possess a phenotype negative for SSEA-1 and positive for SSEA-3, SSEA-4, TRA-1-60, TRA-1-81, and alkaline phosphatase.

8. The method of claim 1, wherein said mesenchymal stem cell are derived from tissue selected from the group consisting of: a) Wharton's Jelly/umbilical cord tissue/peri-natal; b) bone marrow; c) peripheral blood; d) mobilized peripheral blood; e) endometrium; f) hair follicle; g) deciduous tooth; h) testicle; i) adipose tissue; j) skin; k) amniotic fluid; l) cord blood; m) omentum; n) muscle; o) amniotic membrane; o) periventricular fluid; and p) placental tissue.

9. The method of claim 8, wherein said mesenchymal stem cells express a marker or plurality of markers selected from the group consisting of: STRO-1, CD90, CD73, CD105, CD54, CD106, HLA-I markers, vimentin, ASMA, collagen-1, fibronectin, LFA-3, ICAM-1, PECAM-1, P-selectin, L-selectin, CD49b/CD29, CD49c/CD29, CD49d/CD29, CD61, CD18, CD29, thrombomodulin, telomerase, CD10, CD13, STRO-2, VCAM-1, CD146, and THY-1.

10. The method of claim 1, wherein said mesenchymal stem cell are activated by exposure to a toll like receptor agonist.

11. The method of claim 10, wherein said activator of TLR-1 is Pam3CSK4.

12. The method of claim 10, wherein said activator of TLR-2 is HKLM.

13. The method of claim 10, wherein said activator of TLR-3 is Poly:IC.

14. The method of claim 10, wherein said activator of TLR-4 is Methadone.

15. The method of claim 10, wherein said activator of TLR-4 is Cocaine.

16. The method of claim 10, wherein said activator of TLR8 is ssRNA40/LyoVec.

17. The method of claim 1, wherein the cells are derived in xenofree media.

18. The method of claim 1, wherein the cells are delivered in platelet rich plasma/platelet lysate carrier solution.

19. The method in claim 1, wherein the cells are functionally activated with a trivalent gene construct.

20. The method in claim 1, wherein the cells are functionally activated with a polyvalent gene construct.
